(19) United States
(12) Patent Application Publication
(10) Pub. No.: US 2011/0008796 A1
(43) Pub. Date: Jan. 13, 2011

(54) INDUSTRIAL METHOD FOR THE ENCAPSULATION OF BIOLOGICAL MATERIAL FOR THE PURPOSE OF STORAGE AT AMBIENT TEMPERATURE WITH A VACUUM SEAL TEST OF THE ENCAPSULATION

(75) Inventors: Sophie Tuffet, Bordeaux (FR); David Georges De Souza, Bordeaux (FR)

Correspondence Address: YOUNG & THOMPSON, 209 Madison Street, Suite 500, Alexandria, VA, 22314, US

(73) Assignee: IMAGENE, S.A. A DIRECTOIRE, PESSAC (FR)

(21) Appl. No.: 12/921,992

(22) PCT Filed: Mar. 10, 2009
(86) PCT No.: PCT/FR09/50394

§ 371 Date: Sep. 10, 2010

(30) Foreign Application Priority Data
Mar. 11, 2008 (FR) 0851565

(51) Int. Cl. C12Q 1/68; G01N 33/50

(52) U.S. Cl. 435/6; 436/94

(57) ABSTRACT

A method for the preparation of a sample of biological material in a container for the purpose of its storage, its recovery, and its subsequent use, includes the succession of the following stages:
preparation of the biological material with solubilization,
quality control of the thus prepared biological material,
labeling of the container with recording of the identification,
introduction of this biological material into the container,
dehydration of the biological material,
sealing of the container,
sealing test, and
placement in a storage station. A method for using the prepared sample is also described.

This invention relates to an industrial method for encapsulation of biological material, in particular DNA, more particularly for the purpose of storage at ambient temperature.

The Patent EP 1 075 515 that describes a method for prolonged storage of DNA in an airtight and rustproof metal capsule is known.

This DNA is encapsulated in a neutral atmosphere and with a very low hygrometric degree so as to make storage possible at ambient temperature, thus preventing the use of complex and costly refrigeration and/or freezing means.

This method can be applied to any biological material of human, animal or plant origin and comprises in particular: tissues; cells; microorganisms such as bacteria, mushrooms, and monocellular algaes; viruses; proteins; and nucleic acids such as DNA and RNA.

The biological material is used for increasingly numerous applications both in the field of research and in numerous other fields such as biotechnology, health, the environment, farm produce, identification, justice, and criminology, and it is becoming imperative to constitute banks of samples of biological material or libraries.

A bank of samples requires absolutely strict classification and identification.

Actually, a possible manual treatment on the laboratory scale is ill-suited to the creation of such libraries.

It is therefore advisable to design automated systems for the production of samples of biological material, starting from the initial biological material until it is used. This use can occur after a very long amount of time, up to several decades later.

Such a system is to provide the following stages: preparation of the sample of biological material and monitoring of its quality, putting said sample into a container and storing it, and then recovery of the biological material for the purpose of its use starting from the sample of biological material that is thus stored.

During all of these stages, it is necessary to ensure the management of the quality and the traceability of all of the samples.

For this purpose, it is necessary to provide an identical container for all of the samples so as to allow handling performed by the different automatic devices and the exchanges of samples between users, for example.

It is also necessary to ensure the storage at ambient temperature under conditions that limit the degradation of biological material and to allow a labeling for the purpose of listing these samples and classifying them while reaching the primary goal: making possible the recovery of the biological material and its use.

This container is to allow handling by the method according to the invention, in an automated manner, and in particular using test tube rack-type supports that comply with the format of the microplates in the so-called SBS standard, a trademark filed by the SBS Company.

The method according to this invention consists in using this container and providing a succession of stages so as to treat the biological material industrially.

The method according to this invention is now described in detail according to a particular and optimized embodiment.

The method for preparing samples of biological material according to this invention in a container for the purpose of its storage, its recovery, and its subsequent use consists in implementing the succession of the following stages:
Preparation of the biological material with solubilization,
Quality control of the thus prepared biological material,
Labeling of the container and putting it on a test tube rack with recording of the identification of the test tube rack and coordinates of the container in the test tube rack,
Introduction of this biological material into each dedicated metal container,
Dehydration of the biological material by being placed under vacuum,
Sealing of the container by a stopper under controlled atmosphere,
Welding of the stopper to the container without thermal heating,
Sealing test by being placed under vacuum and detection of gas particles from the controlled atmosphere, and
Placement in a storage station.

The method according to this invention also relates to the use of the thus prepared sample according to the preceding stages.

This method for use consists of the succession of the following stages:
Identification of a determined container placed on a test tube rack by reading the labeling,
Opening by perforation of the stopper of the container,
Resolubilization of the biological material in the container,
Sampling of the recovered biological material, and
Use in particular for purposes of analysis.

The invention is now described in detail.

The method according to this invention therefore consists in preparing the biological material, purifying it and solubilizing it.

If necessary, this biological material is distributed into several samples of smaller volume based on requirements and the initial volume of the available material; it is the aliquoting operation during which or prior to which the quality of the sample is controlled. In addition, each sample and/or each aliquot is listed.

The quality control of the biological material is, for example, the combination of techniques such as a measurement of optical density, a measurement of fluorescence, an electrophoresis gel, a metering of proteins, or a molecular amplification. Each sample that is controlled and that meets the quality criteria is to be introduced into a container.

The parameters that relate to this container itself are very important.

For the implementation of the method, the container that is adopted is metal and cylindrical in shape, obtained by deformation of metal, of the deep drawing type. The metal is advantageously stainless steel 304L or, to be more metallurgically correct, indexed under the grade Z2CN18-10.

The dimensions of this container, in this case a diameter of 7 mm, a length of 18 mm of the cylindrical envelope, and a wall thickness of 0.25 mm, are selected for numerous reasons.

First of all, these dimensions are defined so that each container can be accommodated in the well of a standard microplate, in particular a microplate with the SBS standard.

In addition, the stainless steel grade makes possible, in addition to the inherent qualities linked to the material such as the resistance to corrosion, on the one hand, an excellent deformability allowing a production of this type of container by deep drawing with a very great precision and a total reproducibility and this at a cost that is quite advantageous, and, on the other hand, a good capacity for welding.

Of course, a cylindrical container could be obtained from a cylinder and a first welded stopper, but this embodiment would only be a technical equivalent. It would have the drawback of requiring an additional weld and would exhibit an additional risk of leakage; it is for this reason that this invention adopts a container that is obtained in a monolithic manner from a single part that is deformed mechanically.

In addition, the container is to be indexed before accommodating the biological material in the sense where it proves necessary in an automated method such as that of this invention to allow total traceability.

This labeling is preferably done by means of a laser. The latter can carry out labeling only by contrast at the surface of the material by a change in state of this surface, readily visible to the naked eye and primarily for any electronic eye. It is also possible to engrave it, i.e., to attack the material.

More particularly, the labeling is positioned on the outside bottom of the container and comprises as identifier a Data Matrix matrix code and/or a system of alpha-numeric characters.

Another solution consists in using an RFID tag, using the radiofrequency waves to identify each container and to add other information to it if necessary.

It should be noted that such an added labeling comprises a tag that ensures the protection of the RFID antenna, whereby the antenna is the active element and is therefore protected. This labeling can be considered permanent.

The thus labeled container is positioned on a test tube rack at a location of which the coordinates are also recorded, in addition to those of the test tube rack.

The container is ready to accommodate the previously identified biological material although an automatic device can deposit the biological material in the indexed container in such a way as to use the references associated with the biological material and with the container that accommodates it.

In the case of the DNA, the liquid in which it is dissolved is preferably introduced into a glass insert that is itself positioned in advance in the container.

The container and the biological material that it has accommodated are subjected to dehydration means of the evaporator-concentrator type. Known means consist in using a placement under vacuum so as to dehydrate the biological material that then forms a dry deposit on the glass insert.

This dehydration is pushed so as to carry the biological material to a suitable dehydration level to prevent any degradation by water, on the order of 1% to provide an order of magnitude in the case of the DNA.

The container and the dehydrated biological material are then subjected to a controlled atmosphere, for example an inert gas or a mixture of inert gases—argon, helium, dry gases—to prevent any reaction of chemical or enzymatic hydrolysis and oxidation.

The container accommodates a stopper so as to imprison the inert gas in the container, thus preventing the presence of oxygen and atmospheric water and sheltering the biological material from the light that can induce modifications in the case of DNA, for example.

This stopper is advantageously cylindrical, of short length, 3 mm for example, provided to be introduced into the cylindrical envelope. Thus, once introduced, the peripheral edge of the stopper is juxtaposed on the peripheral edge of the container. This is the fitting of a cylinder into a cylinder.

Simultaneously, under controlled atmosphere, the stopper is welded on the container so as to make the container airtight and to guarantee that the dehydrated biological contents are held under controlled atmosphere and protected from light.

The weld is a weld without the addition of metal and without the addition of heat of the weld type by laser beam or electron beam, whereby the laser beam is preferred for its simple use and its cost. In addition, and this is very important, the weld by laser beam is preferably of the pulsed YAG laser type, which very greatly limits the rise in temperature outside of the welding zone, to the point of considering it negligible relative to the container. The small thickness of the walls also requires a limited power and also a very short intervention time.

The words “without the addition of heat” mean that the heat that is released is localized and very weak and that the biological material does not undergo any degradation, all the more so if it is placed in a glass insert that partially isolates it from the metal container.

The method thus makes it possible to seal the containers very quickly and reliably in an airtight manner and automatically since the diameter is constant and perfectly reproduced.

Nevertheless, so as to verify that the container is quite airtight and that the biological material will preserve its integrity, the method according to the invention provides a systematic test of sealing against gases after welding.

This test consists in placing the welded container in a chamber that is placed under vacuum, a chamber into which is placed a sensor that can detect the gas or the gas mixture of the controlled atmosphere that is introduced and imprisoned in the container.

In the case of the sealing defect of the weld, molecules of the gas or gases imprisoned in the container will escape from the chamber during the creation of a partial vacuum and will be detected by the sensor, leading to a downgrading of the sample, with the result that the defective container will be removed from the system.

The biological material can be recovered and replaced in a new container.

In the case where the leakage test proves satisfactory, the container that is sealed in an airtight manner is repositioned on its test tube rack, and the different containers can be stored in a library, for example.

Then, indexed test tube racks of the microplate type are placed in each of the wells from which an indexed container and its biological material, also indexed, are found.

It is therefore easy to classify these thus produced samples.

The storage is done at ambient temperature for most of the samples.

Nevertheless, using the method according to the invention, it is noted that certain samples of fragile biological material, which it was suitable to store at freezing temperatures of lower than or equal to −20° C., can now be stored at temperatures that are close to zero by the implementation of the method according to the invention, which is a considerable asset over very long storage periods.

The method according to this invention also provides for the recovery and the use of this biological material since it is the very essence of this invention: store in order to use.

For this purpose, the method provides for means for resolubilization in a solvent of biological material in such a way as to allow its use for purposes of analyses, for example.

The method therefore provides for the collection of the sample or samples that are researched and listed in the library.

Once the sample is identified, the container can be opened by simple perforation of the stopper. This perforation advantageously prevents the introduction of metal and other particles that could be obtained from an opening by mechanical machining. In addition, by a shape memory effect linked to the deep drawing that is carried out, the cut parts curve by allowing a perfectly detached opening that is accessible to sampling means such as automated pipettes.

The position of the perforation is perfectly controlled and centered in the stopper although the movements of the pipettes of the automatic devices can be perfectly programmed.

The different stages of the method as described can be carried out successively or in parallel. Thus, the labeling of the container can be done parallel to the preparation of the biological material in such a way that the material can be introduced into the thus labeled dedicated container.

The labeling could also be done prior to the preparation of the material according to the organization of the system.

1. Method for the preparation of a sample of biological material in a container for the purpose of its storage, its recovery, and its subsequent use, characterized in that it comprises the succession of the following stages:
Preparation of the biological material with solubilization,
Quality control of the thus prepared biological material,
Labeling of the container with recording of the identification,
Introduction of this biological material into said container,
Dehydration of the biological material,
Sealing of the container under controlled atmosphere,
Sealing test of the container by placing the sealed container in a chamber that is placed under vacuum, and by detecting the gas or the mixture of gas from the controlled atmosphere that is introduced and imprisoned in the container that escapes from the container under the action of vacuum, and
Placement in a storage station when the test is favorable.

2. Method for preparation of a sample of biological material according to claim 1, wherein the container is metal and comprises a stopper, and the sealing is ensured by welding this stopper without thermal heating.

3. Method for preparation of a sample of biological material according to claim 2, wherein the welding is carried out by welding using a laser beam of the pulsed YAG laser type.

4. Method for preparation of a sample of biological material according to claim 3, wherein the controlled atmosphere is composed of an inert gas or a mixture of dry inert gases to prevent any reaction of chemical or enzymatic hydrolysis and oxidation of said biological material.

5. Method for the preparation of a sample of biological material according to claim 1, wherein the placement in a storage station provides for the placement of each container on a test tube rack, an identification of the test tube rack, and a recording of the position of said container on the test tube rack.

6. Method for the preparation of a sample of biological material according to claim 1, wherein dehydration is done by being placed under vacuum.

7. Method for using a sample that is prepared by the implementation of the method according to claim 1, wherein it comprises the following stages:
Identification of a determined container placed on a test tube rack by reading the labeling,
Opening by perforation of the stopper of the container,
Resolubilization of the biological material in the container,
Sampling of the thus recovered biological material, and
Use of the biological material sampled in particular for purposes of analysis.
